(19) United States
(12) Patent Application Publication
(10) Pub. No.: US 2026/0100254 A1
(43) Pub. Date: Apr. 9, 2026

(54) GENERATING THREE-DIMENSIONAL MOLECULE STRUCTURES USING GENERATIVE ARTIFICIAL INTELLIGENCE MODELS

(71) Applicant: NVIDIA Corporation, Santa Clara, CA (US)

(72) Inventors: Daniel Alexander Reidenbach, San Jose, CA (US); Filipp Nikitin, Pittsburgh, PA (US)

(21) Appl. No.: 19/180,012

(22) Filed: Apr. 15, 2025

Related U.S. Application Data
(60) Provisional application No. 63/703,798, filed on Oct. 4, 2024.

(51) Int. Cl. G16C 20/50; G06N 3/0475; G16C 20/30; G16C 20/70

(52) U.S. Cl. CPC G16C 20/50; G06N 3/0475; G16C 20/30; G16C 20/70

(57) ABSTRACT

In various examples, methods for generating accurate three-dimensional molecular structures using a generative artificial intelligence model include receiving a request to generate a molecular structure, the request including an initial structure, atom type information, and bond type information for the molecular structure; generating a fused input feature based on embedding representations of the initial structure, the atom type information, and the bond type information; generating an intermediate predicted molecular structure based on a transformer layer of a generative artificial intelligence model and the fused input feature; refining, using a graph neural network-based layer in the generative artificial intelligence model, the intermediate predicted molecular structure into the molecular structure; and outputting the molecular structure.

### CROSS-REFERENCE TO RELATED APPLICATIONS

This application claims priority to and benefit of U.S. Patent Application Ser. No. 63/703,798, entitled “3D Molecule Generation,” filed Oct. 4, 2024, and assigned to the assignee hereof, the entire contents of which are hereby incorporated by reference.

### TECHNICAL FIELD

Embodiments of the present disclosure relate generally to three-dimensional molecule generation using generative artificial intelligence models.

### BACKGROUND

Molecules are structures defined by molecular bonds between different atoms in a three-dimensional space. An atom within a molecule may have any number of bonds with other molecules with bond lengths (defined in angstroms (Å)) and bond angles between adjacent bonds. In the molecular virtual screening and design tasks, generating these three-dimensional molecular structures may allow for molecules to be evaluated for their potential as a therapeutic (e.g., a small molecule therapeutic, a large molecule therapeutic (e.g., a biologic product), another protein, etc.). For example, based on three-dimensional molecules generated in the process of virtual screening or drug discovery, affinity predictions between a protein (with an a priori known structure) and a ligand (e.g., a generated three-dimensional molecule that is designed to bond to the protein) can be predicted to determine whether a molecule is a candidate for synthesis and testing for a therapeutic effect relative to the target molecule. For example, molecules with high predicted binding affinities may be molecules with a strong bond in a biological complex (e.g., molecules that are ionically bonded, molecules that are bonded based on a large number of shared electrons in a covalent bond, etc.) and thus may be candidates for synthesis and testing. Meanwhile, molecules with low predicted binding affinities may have weak bonds in a biological complex (e.g., molecules that are bonded by hydrogen bonds, molecules bonded by van der Waals interaction, etc.) and thus may not be candidates for synthesis and testing.

Three-dimensional molecules can be generated de novo using generative artificial intelligence models. For example, a diffusion model can be used to unconditionally generate a three-dimensional molecule from noise using an iterative process. However, generative artificial intelligence models used in generating three-dimensional molecules may not do so efficiently and may not be able to generate valid molecules (e.g., molecules that can be synthesized, conform to rules defining maximum bond distances, bond types, and other chemical structure rules, stable molecules at or near local minima on a potential energy surface, etc.).

As the foregoing illustrates, what is needed in the art are more effective techniques for generating three-dimensional molecules using generative artificial intelligence models.

### BRIEF DESCRIPTION OF DRAWINGS

FIG. 1 illustrates a block diagram of a computing system configured to implement one or more aspects of at least one embodiment;

FIG. 2 illustrates a generative artificial intelligence model configured to generate molecule structures based on input feature fusion, according to at least one embodiment;

FIG. 3 illustrates example operations for generating a molecule structure based on input feature fusion;

FIG. 4A illustrates inference and/or training logic, according to at least one embodiment;

FIG. 4B illustrates inference and/or training logic, according to at least one embodiment; and

FIG. 5 illustrates training and deployment of a neural network, according to at least one embodiment.

### DETAILED DESCRIPTION

As discussed herein, the creation of molecules for testing as potential treatments for various medical conditions is an important task in drug discovery. By creating molecules for evaluation as a potential therapeutic, the process of drug discovery may be accelerated, and resources may be dedicated to testing molecules that are more likely to have a therapeutic effect than those that are less likely to have a therapeutic effect or are likely to have no therapeutic effect at all. As discussed above, however, molecule structure generation using generative artificial intelligence models may be a computationally complex task and may not lead to the generation of valid molecules.

To generate three-dimensional molecules accurate and efficiently, embodiments presented herein provide techniques for using fused input features generated from an input structure and defined structural attributes of a molecule (e.g., atom types, bonds between atoms, atom charge features, etc.) as an input into a generative artificial intelligence model. The generative artificial intelligence model is generally configured to generate an intermediate molecular structure via a transformer layer and to refine the intermediate molecular structure via an equivariant neural network layer, and to do so via at least one (e.g., a plurality of) iterations through the model. The output of the generative artificial intelligence model may be a three-dimensional structure of the molecule and a two-dimensional graph representing the molecule.

One technical advantage of the disclosed techniques relative to prior approaches is increased inferencing speed and molecular structure validity. By using fused inputs of molecular structure and other features of the molecule, embodiments presented herein may generate a significantly larger proportion of valid molecules at large molecule sizes than generated by other three-dimensional generative models. Further, the molecules generated using the generative artificial intelligence models described herein may be generated with smaller energy differences between local minima in an energy landscape (e.g., a ground-truth molecule) and the generated molecule. For example, the energy differences between ground-truth molecules and molecules generated using techniques described herein may be closer to an accuracy threshold value (e.g., a threshold energetic difference) than energy differences between ground-truth molecules and molecules generated using prior techniques.

The above examples are not in any way intended to be limiting. As persons skilled in the art will appreciate, as a general matter, the techniques for automatically generating dialogue flows from unlabeled conversation data can be implemented in any suitable application.

The systems and methods described herein may be used for a variety of purposes, by way of example and without limitation, for use in systems associated with machine control, machine locomotion, machine driving, synthetic data generation, model training, perception, augmented reality, virtual reality, mixed reality, robotics, security and surveillance, simulation and digital twinning, autonomous or semi-autonomous machine applications, deep learning, environment simulation, data center processing, conversational AI, generative AI, light transport simulation (e.g., ray-tracing, path tracing, etc.), collaborative content creation for 3D assets, cloud computing and/or any other suitable applications.

Disclosed embodiments may be comprised in a variety of different systems such as automotive systems (e.g., an infotainment or plug-in gaming/streaming system of an autonomous or semi-autonomous machine), systems implemented using a robot, aerial systems, medial systems, boating systems, smart area monitoring systems, systems for performing deep learning operations, systems for performing simulation operations, systems for performing digital twin operations, systems implemented using an edge device, systems incorporating one or more virtual machines (VMs), systems using or deploying one or more inference microservices, systems that incorporate one or more machine learning models deployed in a service or microservice along with an OS-level virtualization package, systems for performing synthetic data generation operations, systems implemented at least partially in a data center, systems for performing conversational AI operations, systems implementing one or more language models—such as large language models (LLMs), small language models (SLMs), vision language models (VLMs), and/or multi-modal language models that may process text, audio, and/or image data, systems for performing light transport simulation, systems for performing collaborative content creation for 3D assets (e.g., systems or platforms that use universal scene descriptor (USD) data, such as OpenUSD), systems implemented at least partially using cloud computing resources, systems for performing generative AI operations, and/or other types of systems.

### System Overview

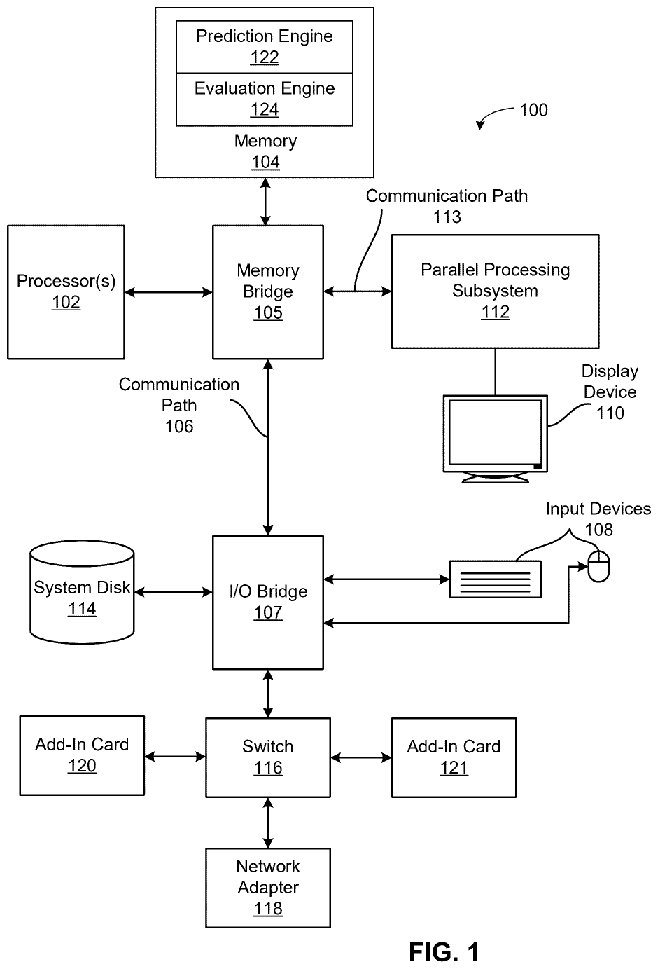

FIG. 1 is a block diagram illustrating a computing system 100 configured to implement one or more aspects of at least one embodiment. In at least one embodiment, computing system 100 may include any type of computing device, including, without limitation, a server machine, a server platform, a desktop machine, a laptop machine, a hand-held/mobile device, a digital kiosk, an in-vehicle infotainment system, a smart speaker or display, a television, and/or a wearable device. In at least one embodiment, computing system 100 is a server machine operating in a data center or a cloud computing environment that provides scalable computing resources as a service over a network.

In various embodiments, computing system 100 includes, without limitation, one or more processors 102 and one or more memories 104 coupled to a parallel processing subsystem 112 via a memory bridge 105 and a communication path 113. Memory bridge 105 is further coupled to an I/O (input/output) bridge 107 via a communication path 106, and I/O bridge 107 is, in turn, coupled to a switch 116.

In one embodiment, I/O bridge 107 is configured to receive user input information from optional input devices 108, such as (but not limited to) a keyboard, mouse, touch screen, sensor data analysis (e.g., evaluating gestures, speech, or other information about one or more uses in a field of view or sensory field of one or more sensors), a VR/MR/AR headset, a gesture recognition system, a steering wheel, mechanical, digital, or touch sensitive buttons or input components, and/or a microphone, and forward the input information to processor(s) 102 for processing. In at least one embodiment, computing system 100 may be a server machine in a cloud computing environment. In such embodiments, computing system 100 may omit input devices 108 and receive equivalent input information as commands (e.g., responsive to one or more inputs from a remote computing device) and/or messages transmitted over a network and received via the network adapter 118. In at least one embodiment, switch 116 is configured to provide connections between I/O bridge 107 and other components of computing system 100, such as a network adapter 118 and various add-in cards 120 and 121.

In at least one embodiment, I/O bridge 107 is coupled to a system disk 114 that may be configured to store content and applications and data for use by processor(s) 102 and parallel processing subsystem 112. In one embodiment, system disk 114 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 107 as well.

In various embodiments, memory bridge 105 may be a Northbridge chip, and I/O bridge 107 may be a Southbridge chip. In addition, communication paths 106 and 113, as well as other communication paths within computing system 100, may be implemented using any technically suitable protocols, including, without limitation, AGP (Accelerated Graphics Port), HyperTransport, or any other bus or point-to-point communication protocol known in the art.

In at least one embodiment, parallel processing subsystem 112 includes a graphics subsystem that delivers pixels to an optional display device 110 that may be any conventional cathode ray tube, liquid crystal display, light-emitting diode display, and/or the like. In such embodiments, parallel processing subsystem 112 may incorporate circuitry optimized for graphics and video processing, including, for example, video output circuitry. Such circuitry may be incorporated across one or more parallel processing units (PPUs), also referred to herein as parallel processors, included within the parallel processing subsystem 112.

In at least one embodiment, parallel processing subsystem 112 incorporates circuitry optimized (e.g., that undergoes optimization) for general purpose and/or compute processing. Again, such circuitry may be incorporated across one or more PPUs included within parallel processing subsystem 112 that are configured to perform such general purpose and/or compute operations. In yet other embodiments, the one or more PPUs included within parallel processing subsystem 112 may be configured to perform graphics processing, general purpose processing, and/or compute processing operations. Memor(ies) 104 include at least one device driver configured to manage the processing operations of the one or more PPUs within parallel processing subsystem 112. In addition, memor(ies) 104 include instructions implementing a prediction engine 122 and an evaluation engine 124, which can be executed by processor(s) and/or parallel processing subsystem 112.

In various embodiments, parallel processing subsystem 112 may be integrated with one or more of the other elements of FIG. 1 to form a single system. For example, parallel processing subsystem 112 may be integrated with processor(s) 102 and other connection circuitry on a single chip to form a system on a chip (SoC).

Processor(s) 102 may include any suitable processor implemented as a central processing unit (CPU), a graphics processing unit (GPU), an application-specific integrated circuit (ASIC), a field programmable gate array (FPGA), an artificial intelligence (AI) accelerator, a deep learning accelerator (DLA), a parallel processing unit (PPU), a data processing unit (DPU), a vector or vision processing unit (VPU), a programmable vision accelerator (PVA) (which may include one or more VPUs, pixel processing engines (PPEs), and/or direct memory access (DMA) systems), any other type of processing unit, or a combination of different processing units, such as a CPU(s) configured to operate in conjunction with a GPU(s). In general, processor(s) 102 may include any technically feasible hardware unit capable of processing data and/or executing software applications. Further, in the context of this disclosure, the computing elements shown in computing system 100 may correspond to a physical computing system (e.g., a system in a data center or a machine) and/or may correspond to a virtual computing instance executing within a computing cloud.

In at least one embodiment, processor(s) 102 issue commands that control the operation of PPUs. In at least one embodiment, communication path 113 is a Peripheral Component Interconnect Express (PCIe) link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processors 102, and the number of parallel processing subsystems 112, may be modified as desired. For example, in at least one embodiment, memor(ies) 104 may be connected to processor(s) 102 directly rather than through memory bridge 105, and other devices may communicate with memor(ies) 104 via memory bridge 105 and processors 102. In other embodiments, parallel processing subsystem 112 may be connected to I/O bridge 107 or directly to processor(s) 102, rather than to memory bridge 105. In still other embodiments, I/O bridge 107 and memory bridge 105 may be integrated into a single chip instead of existing as one or more discrete devices. In certain embodiments, one or more components shown in FIG. 1 may not be present. For example, switch 116 may be eliminated, and network adapter 118 and add-in cards 120, 121 would connect directly to I/O bridge 107. Further, in certain embodiments, one or more components shown in FIG. 1 may be implemented as virtualized resources in a virtual computing environment, such as a cloud computing environment. In particular, the parallel processing subsystem 112 may be implemented as a virtualized parallel processing subsystem in at least one embodiment. For example, the parallel processing subsystem 112 may be implemented as a virtual graphics processing unit(s) (vGPU(s)) that renders graphics on a virtual machine(s) (VM(s)) executing on a server machine(s) whose GPU(s) and other physical resources are shared across one or more VMs.

In some embodiments, prediction engine 122 and evaluation engine 124 include functionality to use machine learning models to generate three-dimensional molecular structures from an input defining the chemical structure features of the requested three-dimensional molecular structure (e.g., constituent atoms, bonds between atoms, atomic charges, etc.) and evaluating the accuracy of the generated molecules relative to a relaxed structure at a local minimum in the energy landscape. A generative artificial intelligence model executed by the prediction engine 122 generates a molecular structure based on generating a fused input for a transformer layer in the generative artificial intelligence model. The fused input, as discussed in further detail herein, may be a set of tokens or other data representing the combination of features associated with an input structure and the chemical structure features of the requested three-dimensional molecular structure. The output of the transformer layer, which may be an intermediate molecular structure, may be input into an equivariant graph neural network for structural refinement, and the refined structure may be output as a three-dimensional molecular structure and a two-dimensional graph representation of the three-dimensional molecular structure. By generating a three-dimensional molecular structure based on a fused input into a generative artificial intelligence model, as discussed, embodiments presented herein may generate higher-quality three-dimensional molecular structures (e.g., a higher proportion of valid molecules) than other generative models. In turn, embodiments presented herein may reduce the amount of computational resources used in generating three-dimensional molecules for further testing, as fewer molecules generated using the techniques described herein may be invalid molecules that are discarded in the process of generating and screening molecules for real-life synthesis and testing.

### Generating Accurate Three-Dimensional Molecules Using Generative Artificial Intelligence Models

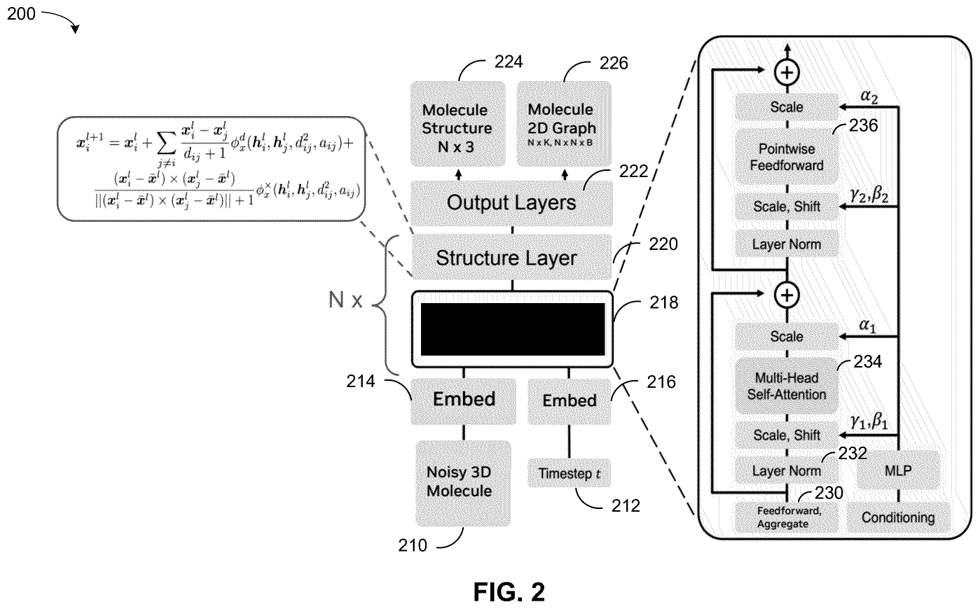

FIG. 2 illustrates a generative artificial intelligence model 200 for generating three-dimensional molecules (also referred to as a conformer) based on a fused input feature generated based on a combination of features associated with an input structure and chemical structure features of a requested three-dimensional molecular structure, according to at least one embodiment. In some embodiments, inferencing operations using the generative artificial intelligence model 200 may be performed by the prediction engine 122 illustrated in FIG. 1.

As illustrated, to generate a three-dimensional molecule, an input of a noisy three-dimensional molecule 210 and a time step t 212 may be input into the generative artificial intelligence model 200. The noisy three-dimensional molecule 210 may include, at an initial step (e.g., at t=0), a noisy input (e.g., an input of Gaussian noise, fixed noise, etc.), as well as information about the chemical structure of the requested three-dimensional molecular structure. At any other inferencing step performed by the generative artificial intelligence model 200, the noisy three-dimensional molecule 210 may be a denoised version of the noisy three-dimensional molecule 210 generated by the generative artificial intelligence model 200 in the previous inferencing round. In some embodiments, a molecule 210 may be defined as M=(X,H,E,C) with N atoms, where X∈ [symbol] ^{N×3} represents the atom coordinates of the three-dimensional molecule, H∈{0,1}^{N×A} represents the atoms in the molecule M, E∈{0,1}^{N×N×B} represents the bonds between the atoms H, and C∈{0,1}^{N×K} represents atomic charge information for the atoms H. In some embodiments, E may be an adjacency matrix in which elements of the matrix corresponding to bonded pairs of atoms have a value of 1 and elements of the matrix corresponding to unbonded pairs of atoms have a value of 0. in some embodiments, X may be modeled as a continuous variable, and H, E, and C may be modeled as discrete variables (e.g., one-hot variables).

Embedding blocks 214 and 216 generate embedding representations of the noisy three-dimensional molecule 210 and the time step t, respectively. These embeddings generally serve as input tokens that the transformer block 218 can use to generate an intermediate molecular structure.

Transformer block 218 may be configured to generate the intermediate molecular structure from a fused embedding representation of the noisy three-dimensional molecule 210 and the embedding representation of the time step by denoising the noisy three-dimensional molecule 210. Transformer block 218 may be, for example, a diffusion-based transformer trained to recover a denoised three-dimensional molecule from a Gaussian distribution or a flow-matching-based transformer trained to transform noise to a denoised three-dimensional molecule based on a continuous vector field that flows from a noise distribution to a target distribution.

In embodiments in which the transformer block 218 is a diffusion block in a generative artificial intelligence model, the transformer block 218 may be trained based on the construction of interpolated (progressively noised) states between exemplars in a training data set and a Gaussian noise distribution [symbol] (xₜ;β(t)x₁,α(t)²x₁), where t∈[0,1] corresponds to a time step where t=1 corresponds to data and t=0 corresponds to noise. In this example,

$${x}_{t} = α ( t ) ϵ + β ( t ) {x}_{1} and$$

$${x}_{1} = \frac{{x}_{t} - α ( t ) ϵ}{β ( t )}$$

where ϵ˜ [symbol] (ϵ;0,I) and x₁˜p_{data}(x₁). The interpolation of states between data at t=1 and [symbol] noise at t=0 may be performed based on variance-preserving differential equation-based noise with a defined noise schedule γₜ, based on a conditional linear vector field with smoothing of a data distribution, or the like. In some embodiments, the transformer block 218 may be trained as a continuous denoising diffusion probabilistic model. In such a model, a gradient-free forward noising process may be performed based on an a priori defined discrete-time variance schedule and a gradient-based denoising process.

In embodiments in which the transformer block 218 is trained based on flow matching techniques, the transformer block 218 may be trained using continuous flow matching techniques to learn a time-dependent vector field v_{θ}(t,xₜ) derived from a differential equation that pushes samples from a noise distribution to a data distribution. The transformer block may be trained based on a loss [symbol] _{CFM} defined as an error between the learned time-dependent vector field v_{θ} and a predicted sample xₜ. The loss [symbol] _{CFM} may be defined by the equation:

$$\begin{matrix}{ℒ}_{CFM} ( θ ) = {𝔼}_{t , ϵ \sim 𝒩 ( ϵ ; 0 , I ) , {x}_{1} \sim {p}_{data ( {x}_{1} )}} {‖ {v}_{θ} ( t , {x}_{t} ) - \frac{d}{dt} {x}_{t} ‖}^{2} \\ = {𝔼}_{t , ϵ \sim 𝒩 ( ϵ ; 0 , I ) , {x}_{1} \sim {p}_{data ( {x}_{1} )}} {‖ {v}_{θ} ( t , {x}_{t} ) - α ( t ) ϵ - β ( t ) {x}_{1} ‖}^{2}\end{matrix}$$

The time-differentiable interpolation discussed above may allow the transformer block 218 to use a probability path that can be easily sampled in learning the time-dependent vector field v_{θ}. In some embodiments, a training engine can simplify training using continuous flow matching techniques based on a data prediction objective. In such a case, an inference Euler differential equation update may be performed after generating a conditional linear vector field v_{θ}, according to the equations:

$${ℒ}_{CFM} ( θ ) = {𝔼}_{t , ϵ \sim 𝒩 ( ϵ ; 0 , I ) , {x}_{1} \sim {p}_{data ( {x}_{1} )}} {‖ {x}_{θ ( t , {x}_{t} )} - {x}_{1} ‖}^{2}$$

$${v}_{θ} ( t , {x}_{t} ) = \frac{{x}_{θ} ( t , {x}_{t} ) - {x}_{t}}{1 - t}$$

$${x}_{t + 1} = {x}_{t} + {v}_{θ} ( t , {x}_{t} ) dt$$

In some embodiments, the transformer block 218 may be trained using discrete diffusion or flow matching techniques. Discrete diffusion may allow for denoising of a noisy input into a predicted molecular structure over a discrete state space. A defined transition matrix may control how the transformer block 218 moves from one discrete state to another discrete state. For a scalar discrete random variable with K categories xₜ, xₜ₋₁∈1, . . . , K, forward transition probabilities may be represented by the matrices |Qₜ|=q(xₜ=j|xₜ₊₁=i). Starting from the initial data point x₁ or the final data point X_{T}, where T corresponds to the total number of discrete time steps, the marginal at step T−t+1 and posterior at time t may be defined by the equations:

$$\begin{matrix}q ( {x}_{t} \vert {x}_{t + 1} ) = Cat ( {x}_{t} ; p = {x}_{t + 1} {Q}_{t} ) , q ( {x}_{t} \vert {x}_{T} ) \\ = Cat ( {x}_{t} ; p - {x}_{T} {\overline{Q}}_{t} )\end{matrix}$$

$$with {\overline{Q}}_{t} = {Q}_{t} {Q}_{t + q} … {Q}_{T}$$

$$\begin{matrix}q ( {x}_{t + 1} \vert {x}_{t} , {x}_{T} ) = \frac{q ( {x}_{t} \vert {x}_{t + 1} , {x}_{T} ) q ( {x}_{t + 1} \vert {x}_{T} )}{q ( {x}_{t} \vert {x}_{T} )} \\ = Cat ( {x}_{t + 1} ; p = \frac{{x}_{t} {Q}_{t}^{T} ⊙ {x}_{T} {\overline{Q}}_{t + 1}}{{x}_{T} {\overline{Q}}_{t} {x}_{t}^{T}} )\end{matrix}$$

In the above equations, Q may be defined as a function of a cosine noise schedule used in continuous denoising diffusion models such that the discrete distribution converges to a desired terminal distribution in T discrete steps.

Finally, in embodiments in which the transformer block 218 is trained using discrete flow matching, the transformer block 218 may be trained to learn conditional flows for the discrete components of a molecular structure (e.g., atom type, bond type, atomic charges, etc.). The discrete flow matching interpolation used in training the transformer block 218 may be defined by the equation:

$${P}_{t \vert 1}^{unif} ( {x}_{t} \vert {x}_{1} ) = q ( {x}_{t} \vert {x}_{1} ) = Cat ( t δ \{ {x}_{1} , {x}_{t} \} + ( 1 - t ) \frac{1}{S}$$

In the above equation, S represents the size of the discrete state space.

The generative artificial intelligence model 200 may generally be configured such that diffusion and flow matching techniques are equivalent in the Gaussian setting. For example, the choices of time distributions and interpolation schedules may allow for a model 200 that uses diffusion techniques to be equivalent to a model 200 that uses flow matching techniques to generate a three-dimensional molecule structure from an input prompt, despite diffusion models and flow matching models using interpolations with different levels of complexity.

Generally, to allow structure, atom type, and bond type prediction to inform each other during molecule generation using the generative artificial intelligence model 200, the generative artificial intelligence model 200 may be trained such that each data type has an independent noise schedule. Two time variables may be sampled by the generative artificial intelligence model 200: t_{continuous} and t_{discrete}. These two time variables may be sampled from the same time distribution. By doing so, embodiments presented herein may interpolate discrete and continuous data within the respective time variable and may allow for independent weighted noise schedules to be used for the different types of data in M. Further, in some embodiments, the generative artificial intelligence model 200 may be trained with self-conditioning. Self-conditioning may be applied to each molecule component in M=(X,H,E,C). The structure component may use an unbiased linear layer and may operate over raw logits.

During inferencing operations, the transformer block 218 aggregates (or fuses) the embeddings of the noisy three-dimensional molecule 210 into a fused input feature at block 230. The fused input feature generally corresponds to a set of input tokens on which self-attention or other operations can be performed to predict a new set of tokens. In some embodiments, block 230 can generate the fused input feature m according to the equation

$$m = \frac{1}{N} {\sum }_{i , j ∈ N} f ( {h}_{norm , i , j} {h}_{norm , i , j} {h}_{n o rm , i , j} , {distance}_{i , j} )$$

where N corresponds to the number of atoms in the molecule M.

In the above equation, hₙₒᵣₘ represents the output of a time-conditioned adaptive layer normalization block 232 for the atom type features included in the noisy three-dimensional molecule 210. eₙₒᵣₘ, meanwhile, represents the output of the time-conditioned adaptive layer normalization block 232 for the edge type features included in the noisy three-dimensional molecule 210. Distance features, distance_{i,j}, may be the concatenation of scalar distances and dot products of distances between the i^{th} and j^{th} atoms in the molecule M.

The fused input feature m may be processed by a multi-headed attention block 234, and the output mha_out of the multi-headed attention block 234 may be processed by a feedforward block 236 to generate Hₒᵤₜ. Hₒᵤₜ generally represents, as discussed, the element types or atoms in the molecule for the intermediate output of the generative artificial intelligence model 200. Meanwhile, to generate Eₒᵤₜ, representing the bond adjacency matrix generated during a denoising process performed by the transformer block 218, the transformer block 218 determines bond adjacency as a function of the outputs of the multi-headed attention block 234 for each pair of atoms i,j, and the output of the multi-headed attention block 234 f(mha_outi, mha_outj) may be processed by the feedforward block 236 to produce Eₒᵤₜ.

Structure layer 220 uses the intermediate molecular structure generated by the transformer block 218 to refine the structure of the three-dimensional molecule generated by the generative artificial intelligence model 200. To do so, structure layer 220 uses an equivariant graph neural network layer with a positional component and a cross-product update component to refine the intermediate molecular structure. The refinement of the intermediate molecular structure performed by the structure layer 220 may, in some embodiments, be performed based on normalizing invariant and equivariant features in the intermediate molecular structure (that is, whether a feature does not change for any given translation of the three-dimensional molecular structure or changes predictably for any given translation of the three-dimensional molecular structure). For invariant features, structure layer 220 normalizes the features based on layer normalization. For equivariant features, structure layer 220 normalizes the features using an E(3) normalization function defined by the equation:

$${x}_{i}^{l + 1} = {x}_{i}^{l} + {\sum }_{j \neq i} \frac{{x}_{i}^{l} - {x}_{j}^{l}}{{d}_{i , j} + 1} {ϕ}_{x}^{d} ( {h}_{i}^{l} , {h}_{j}^{l} , {d}_{i , j}^{2} , {a}_{i j} ) + \frac{( {x}_{i}^{l} - {\overline{x}}^{l} ) \times ( {x}_{j}^{l} - {\overline{x}}^{l} )}{‖ ( {x}_{i}^{l} - {\overline{x}}^{l} ) \times ( {x}_{j}^{l} - {\overline{x}}^{l} ) ‖ + 1} {ϕ}_{x}^{\times } ( {h}_{i}^{l} , {h}_{j}^{l} , {d}_{i , j}^{2} {a}_{i j} )$$

As illustrated, the generative artificial intelligence model 200 may include any number N of transformer block 218-structure layer 220 pairs. Generally, the output of the i^{th} structure layer 220 may be provided as input to the i+1^{th} transformer block 218, where i<N. When i=N (e.g., in the last pairing of transformer block 218 and structure layer 220), the output of structure layer 220 may be provided to output layers 222 for use in generating and outputting the requested molecular structure. The resulting output of output layers 222 may include a three-dimensional molecular structure 224 and a two-dimensional graph representation 226 of the three-dimensional molecular structure 224.

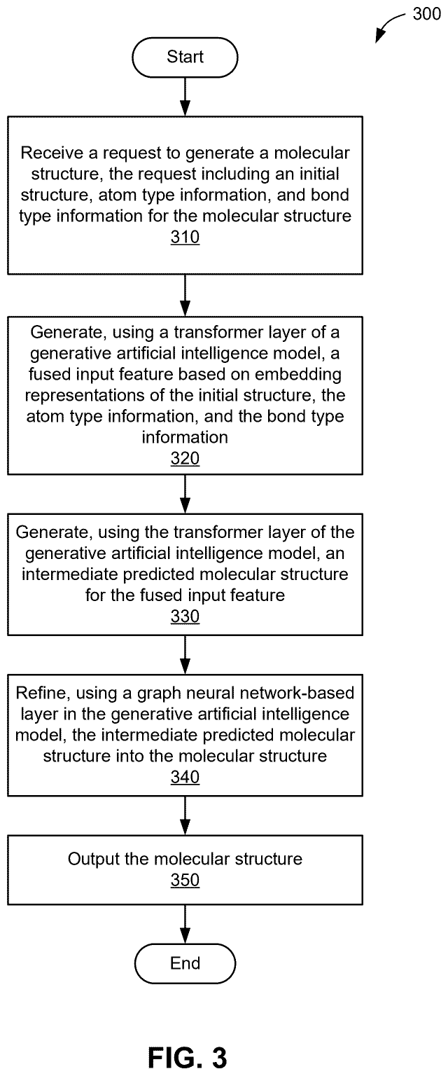

FIG. 3 illustrates example operations 300 for generating a molecule structure based on input feature fusion, according to embodiments of the present disclosure. Operations 300 may be performed, for example, by a prediction engine on which a generative artificial intelligence model is deployed, such as prediction engine 122 illustrated in FIG. 1.

As illustrated, operations 300 begin at block 310, where prediction engine 122 receives a request to generate a molecular structure. Generally, the request includes an initial structure, atom type information, and bond type information for the molecular structure. The initial structure may be a noise distribution (e.g., Gaussian noise, fixed noise, etc.) at an initial time step or a partially denoised input at a time step between an initial time step and a final time step.

The request generally includes a molecule M defined as a tuple of structure, atom type information, and bond type information (e.g., M=(X,H,E,C)). The atom type information may include elemental information and charge information for each atom in the molecular structure. The bond type information may include a bond adjacency matrix. The bond adjacency matrix may be an N×N matrix structured as a one-hot matrix, where element i,j in the bond adjacency matrix E has a value of 1 where element i and element j is bonded in the molecule and a value of 0 where element i an element j is not bonded in the molecule.

Operations 300 proceed to block 320, where prediction engine 122 generates a fused input feature based on embedding representations of the initial structure, the atom type information, and the bond type information. In some embodiments, a transformer layer of a generative artificial intelligence model deployed to prediction engine 122 can generate the fused input feature.

In some embodiments, prediction engine 122 generates the fused input feature based on generating an embedding representation of the initial structure, an embedding representation of the atom type information, and an embedding representation of the bond type information. Prediction engine 122 then aggregates the embedding representation of the initial structure, the embedding representation of the atom type information, and the embedding representation of the bond type information into a fused input that includes a plurality of input tokens. The embedding representations of the initial structure, the atom type information, and the bond type information may be generated by a feedforward neural network.

In some embodiments, the plurality of input tokens may serve as an input into transformer layers of the generative artificial intelligence model. In some embodiments, the fused input feature may further be based on a time variable associated with a number of inferencing rounds previously performed with respect to the request.

At block 330, prediction engine 122 generates an intermediate predicted molecular structure for the fused input feature. Generally, prediction engine 122 generates the intermediate predicted molecular structure using the transformer layer of the generative artificial intelligence model and the fused input feature.

In some embodiments, the transformer layer of the generative artificial intelligence model may be a diffusion layer configured to generate the intermediate predicted molecular structure based on denoising a noisy representation of the molecular structure.

In some embodiments, the transformer layer of the generative artificial intelligence model may be a flow-matching layer.

At block 340, prediction engine 122 refines, using a graph neural network-based layer in the generative artificial intelligence model, the intermediate predicted molecular structure into the molecular structure.

In some embodiments, the graph neural network-based layer may be configured to refine the intermediate predicted molecular structure based on positional update and a cross-product update. The intermediate predicted molecular structure may be refined into, for example, a three-dimensional conformer and a two-dimensional graph representation of the three-dimensional structure.

In some embodiments, operations 300 include prediction engine 122 receiving feedback data based on results of the experimental evaluation of the molecular structure. The feedback data may indicate, for example, whether the molecular structure is a valid or an invalid structure, specific details of why the molecular structure is a valid or invalid structure (e.g., invalid bond distances or charges), and so on. Operations 300 may further include prediction engine 122 updating one or more parameters of the generative artificial intelligence model based on the feedback data to improve accuracy of subsequent molecular structure generation.

At block 350, prediction engine 122 outputs the molecular structure. In some embodiments, the molecular structure is displayed on a graphical user interface for further analysis and visualization. For example, the molecule structure may be output as part of a drug discovery pipeline, where it can be selected for synthesis and experimental testing or evaluation to evaluate its potential as a treatment for one or more target medical conditions. In certain cases, the structure may be chosen for in vitro validation as a candidate therapeutic for a specific disease, such as Alzheimer's disease. In some embodiments, the generative intelligence model may be updated based on results of the testing to improve accuracy of subsequent molecular structure predictions.

### Benchmarks for Evaluating the Quality of Three-Dimensional Molecules Generated Using Generative Artificial Intelligence Models

Coverage and average minimum root mean square deviation (RMSD) (AMR) measurements between a ground-truth molecule and a generated molecule are techniques that can be used to evaluate the quality of a ground-truth molecule. Coverage generally refers to a percentage of generated three-dimensional molecules have a minimum error under a specified AMR threshold. Meanwhile, recall generally refers to a measurement of the overall spatial accuracy of each generated three-dimensional molecule in a test set. Using the coverage and recall metrics, the techniques described herein allow for the generation of three-dimensional molecular structures with similar performance relative to other techniques that use computationally expensive approximations of quantum chemistry to establish bond lengths and angles. However, it should be recognized that the generative models discussed herein are able to create new molecules instead of conformers for a given formulation of a molecule, and thus, coverage and AMR metrics may not provide usable information on the quality of the molecules generated using the techniques described herein (e.g., based on a desired two-dimensional topology of atoms and bonds between atoms).

To determine the quality of a three-dimensional molecule generated using generative artificial intelligence models, embodiments presented herein provide techniques for comparing a generated three-dimensional molecule to a ground-truth molecule (which may be a molecule corresponding to a local minimum in the potential energy surface or some other target molecule). A generated molecule may fail to conform to ground-truth molecule for various reasons. For example, bond orders may be incorrect, the geometry of the generated molecule may be distorted, atomic charge information may be incorrect, and the like. Thus, to benchmark the quality of a generated three-dimensional molecule and its corresponding ground-truth molecule, embodiments presented herein provide techniques for evaluating the geometric fidelity of a generated molecule to its relaxed counterpart at a local minimum in the potential energy surface.

One metric for benchmarking the quality of a generated molecule may be defined as a distance between the generated distribution and target distribution of bond angles in the generated and ground-truth molecules. The distance may be the Wasserstein distance between bond angles in the generated distribution and target distribution of molecules, defined by the equation:

$${W}_{a n g l e s} = {\sum }_{y ∈ atom types} p ( y ) \cdot {W}_{1} ( {\hat{D}}_{a n g l e} ( y ) , {D}_{a n g l e} ( y ) )$$

where p(y) is the probability of atom type y, W₁ denotes the Wasserstein distance, \hat{D}_{angle}(y) is the bond angle distribution for atom type y in the generated molecule set, and D_{angle}(y) is the corresponding distribution of bond angles in a test data set.

Another metric for benchmarking the quality of a generated molecule may be based on torsion angles of atoms in a molecule. The torsion angle may describe, for example, the twist or rotation around a bound of a set of atoms. The distance between torsion angles in the generated distribution and target distribution of molecules may be defined by the equation:

$${W}_{torsions} = {\sum }_{y ∈ bond types} p ( y ) \cdot {W}_{1} ( {\hat{D}}_{torsion} ( y ) , {D}_{torsion} ( y ) )$$

where p(y) is the probability of bond type y, W₁ denotes the Wasserstein distance, Dₜₒᵣₛᵢₒₙ(y) is the torsion angle distribution for bond type y in the generated molecule set, and Dₜₒᵣₛᵢₒₙ(y) is the corresponding distribution of torsion angles in a test data set.

Still further metrics can be defined to quantify the quality of generated three-dimensional molecules relative to a relaxed (or presumed ground-truth) molecule associated with a local minimum in a potential energy surface. In some embodiments, a difference in bond length between two atoms i and j can be compared between a generated molecule and a ground-truth molecule. Given

$${r}_{ij}^{init}$$

as the distance between atoms i and j in the generated molecule and

$${r}_{ij}^{opt}$$

as the distance between atoms i and j in the relaxed molecule, the bond length difference may be defined by the equation:

$$Δ {r}_{i j} = \vert {r}_{ij}^{init} - {r}_{ij}^{opt} \vert$$

The average bond length difference and frequencies for each combination of source atom type, bond type, and target atom type may be computed. A final metric may be the weighted sum of the average differences for each combination of source atom type, bond type, and target atom type to determine an overall bond length difference between generated and relaxed molecules.

In some embodiments, a difference in bond angle between atoms i, j, and k may be used as a matric for evaluating the quality of generated three-dimensional molecules relative to their relaxed (or presumed ground-truth) molecule counterparts. Given

$${θ}_{ijk}^{init}$$

as the bond angle at atom j between atoms i and k in the generated molecule and

$${θ}_{ijk}^{opt}$$

as the bond angle at atom j between atoms i and k in the relaxed molecule, the bond angle difference may be defined by the equation:

$$Δ {θ}_{ijk} = min ( \vert {θ}_{ijk}^{init} - {θ}_{ijk}^{opt} \vert , 180 ° - \vert {θ}_{ijk}^{init} - {θ}_{ijk}^{opt} \vert )$$

The average bond angle difference may be computed and grouped based on the types of atoms and bonds involved, and a final metric may be the weighted sum of the average bond angle differences calculated for the set of generated molecules.

In some embodiments, torsion angle differences for sets of atoms i, j, k, l in the generated three-dimensional molecules and the corresponding relaxed molecules may be used as a matric for evaluating the quality of the generated three-dimensional molecules relative to their relaxed (or presumed ground-truth) molecule counterparts. Given

$${ϕ}_{ijkl}^{init}$$

as the dihedral angle in a generated molecule and

$${ϕ}_{ijkl}^{opt}$$

as the dihedral angle in a relaxed molecule, the difference in torsion angles between a generated molecule and a relaxed molecule may be defined by the equation:

$$Δ {ϕ}_{ijkl} = min ( \vert {ϕ}_{ijkl}^{init} - {ϕ}_{ijkl}^{opt} \vert , 360 ° - \vert {ϕ}_{ijkl}^{init} - {ϕ}_{ijkl}^{opt} \vert )$$

The equation above may account for the periodicity of dihedral angles such that the smallest difference between torsion angles is used to define the torsion angle difference between generated and relaxed molecules.

In some embodiments, energy benchmarks may also be used to determine the quality of a generated molecule relative to a relaxed molecule in the potential energy surface. Generally, molecules at local minima in a potential energy surface may be considered accurate molecules because molecules at the local minima are generally stable molecules while those not at the local minima in the potential energy surface may not be stable molecules. A relaxation energy ΔEᵣₑₗₐₓ may be defined as the energy difference between a relaxation energy Eₒₚₜ for the relaxed molecule at a local minimum in a potential energy surface and a relaxation energy Eᵢₙᵢₜ for the generated molecule, according to the equation:

$$Δ {E}_{relax} = {E}_{opt} - {E}_{init}$$

A mean and a median relaxation energy may be used to determine, from an energetic standpoint, the quality of generated molecules. A ΔEᵣₑₗₐₓ closer to 0 for a molecule may generally signify that the generated molecule is closer to a local minimum in the potential energy surface than a ΔEᵣₑₗₐₓ further away from 0.

Finally, it should be noted that three-dimensional molecules exist in dynamic equilibrium, with population distribution determined by the relative free energies between different conformers associated with the three-dimensional molecules. An equilibrium constant K between two conformers may be defined by the equation:

$$K = {e}^{- Δ G ° / RT}$$

where ΔG° corresponds to a standard free energy difference, R is the gas constant, and T is the temperature in Kelvin. At a temperature of 298 K (˜25° Celsius), the thermal energy RT may b approximately 0.6 kcal/mol. A free energy difference ΔG° of 1.36 kcal/mol may correspond to a tenfold difference in the equilibrium constant. Because the equilibrium constant may grow exponentially, low-energy conformers corresponding to relaxed (or presumed ground-truth) conformers may be defined as those within a 2.5 kcal/mol energy window.

The three-dimensional molecules generated using the techniques described herein may have a median relaxation energy ΔEᵣₑₗₐₓ around 3 kcal/mol, approaching the thermodynamically relevant interval. Thus, by using a fused input of molecular structure features, atomic information features, and bond information features for a molecule to be generated by a generative artificial intelligence model, embodiments presented herein may allow for improved accurate and reduced computational resource expenditure in generating three-dimensional molecules. For example, the molecules generated using the techniques described herein may be energetically feasible and thus may be accurate molecules that can be used in various applications where the accuracy of three-dimensional molecule structures has a significant impact on downstream tasks, such as conformation searches, drug discovery, binding affinity prediction, and the like. Further, the molecules generated using the techniques described herein may have smaller bond length, bond angle, and dihedral differences relative to molecules at local minima in the potential energy surface than molecules generated using other techniques. These smaller bond length, bond angle, and dihedral differences further show that molecules generated using the techniques described herein may be more accurate and conform more closely to ground-truth molecules (or molecules at local minima in the potential energy surface) than molecules generated using other techniques.

### Inference and Training Logic

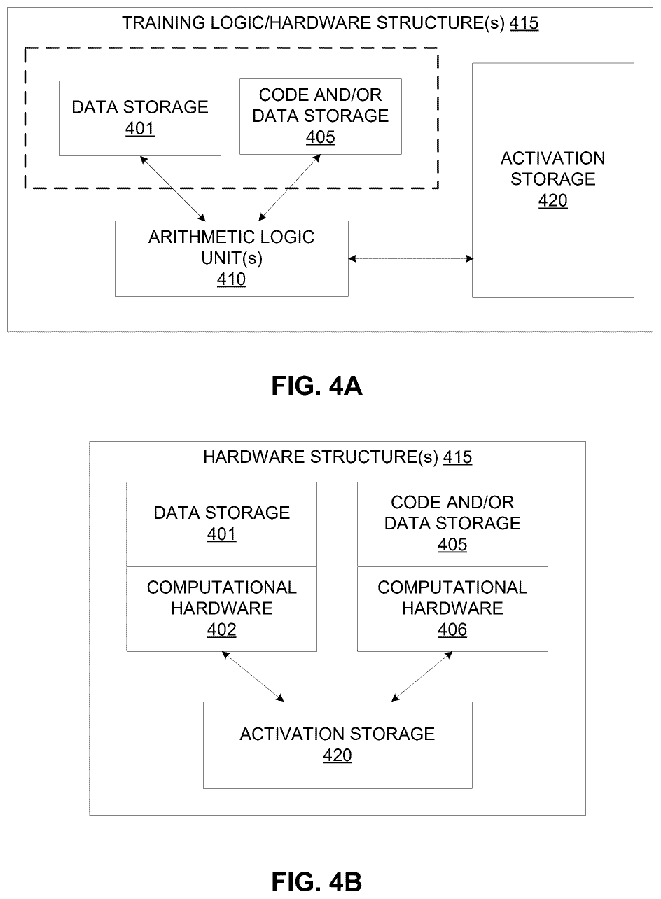

FIG. 4A illustrates inference and/or training logic 415 used to perform inferencing and/or training operations associated with one or more embodiments. Details regarding inference and/or training logic 415 are provided herein in conjunction with at least FIGS. 4A and/or 4B.

In at least one embodiment, inference and/or training logic 415 may include, without limitation, code and/or data storage 401 to store forward and/or output weight and/or input/output data, and/or other parameters to configure neurons or layers of a neural network trained and/or used for inferencing in aspects of one or more embodiments. In at least one embodiment, training logic 415 may include, or be coupled to code and/or data storage 401 to store graph code or other software to control timing and/or order, in which weight and/or other parameter information is to be loaded to configure, logic, including integer and/or floating point units (collectively, arithmetic logic units (ALUs)). In at least one embodiment, code, such as graph code, loads weight or other parameter information into processor ALUs based on an architecture of a neural network to which such code corresponds. In at least one embodiment, code and/or data storage 401 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 401 may be included with other on-chip or off-chip data storage, including a processor's L1, L2, or L3 cache or system memory.

In at least one embodiment, any portion of code and/or data storage 401 may be internal or external to one or more processors or other hardware logic devices or circuits. In at least one embodiment, code and/or code and/or data storage 401 may be cache memory, dynamic randomly addressable memory (“DRAM”), static randomly addressable memory (“SRAM”), non-volatile memory (e.g., flash memory), or other storage. In at least one embodiment, a choice of whether code and/or code and/or data storage 401 is internal or external to a processor, for example, or comprising DRAM, SRAM, flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 415 may include, without limitation, a code and/or data storage 405 to store backward and/or output weight and/or input/output data corresponding to neurons or layers of a neural network trained and/or used for inferencing in aspects of one or more embodiments. In at least one embodiment, code and/or data storage 405 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 415 may include, or be coupled to code and/or data storage 405 to store graph code or other software to control timing and/or order, in which weight and/or other parameter information is to be loaded to configure, logic, including integer and/or floating point units (collectively, arithmetic logic units (ALUs)).

In at least one embodiment, code, such as graph code, causes the loading of weight or other parameter information into processor ALUs based on an architecture of a neural network to which such code corresponds. In at least one embodiment, any portion of code and/or data storage 405 may be included with other on-chip or off-chip data storage, including a processor's L1, L2, or L3 cache or system memory. In at least one embodiment, any portion of code and/or data storage 405 may be internal or external to one or more processors or other hardware logic devices or circuits. In at least one embodiment, code and/or data storage 405 may be cache memory, DRAM, SRAM, non-volatile memory (e.g., flash memory), or other storage. In at least one embodiment, a choice of whether code and/or data storage 405 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 401 and code and/or data storage 405 may be separate storage structures. In at least one embodiment, code and/or data storage 401 and code and/or data storage 405 may be a combined storage structure. In at least one embodiment, code and/or data storage 401 and code and/or data storage 405 may be partially combined and partially separate. In at least one embodiment, any portion of code and/or data storage 401 and code and/or data storage 405 may be included with other on-chip or off-chip data storage, including a processor's L1, L2, or L3 cache or system memory.

In at least one embodiment, inference and/or training logic 415 may include, without limitation, one or more arithmetic logic unit(s) (“ALU(s)”) 410, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 420 that are functions of input/output and/or weight parameter data stored in code and/or data storage 401 and/or code and/or data storage 405. In at least one embodiment, activations stored in activation storage 420 are generated according to linear algebraic and or matrix-based mathematics performed by ALU(s) 410 in response to performing instructions or other code, wherein weight values stored in code and/or data storage 405 and/or data storage 401 are used as operands along with other values, such as bias values, gradient information, momentum values, or other parameters or hyperparameters, any or all of which may be stored in code and/or data storage 405 or code and/or data storage 401 or another storage on or off-chip.

In at least one embodiment, ALU(s) 410 are included within one or more processors or other hardware logic devices or circuits, whereas in another embodiment, ALU(s) 410 may be external to a processor or other hardware logic device or circuit that uses them (e.g., a coprocessor). In at least one embodiment, ALUs 410 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 401, code and/or data storage 405, and activation storage 420 may share a processor or other hardware logic device or circuit, whereas in another embodiment, they may be in different processors or other hardware logic devices or circuits, or some combination of same and different processors or other hardware logic devices or circuits. In at least one embodiment, any portion of activation storage 420 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 420 may be cache memory, DRAM, SRAM, non-volatile memory (e.g., flash memory), or other storage. In at least one embodiment, activation storage 420 may be completely or partially within or external to one or more processors or other logical circuits. In at least one embodiment, a choice of whether activation storage 420 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 415 illustrated in FIG. 4A may be used in conjunction with an application-specific integrated circuit (“ASIC”), such as a TensorFlow® Processing Unit from Google, an inference processing unit (IPU) from Graphcore™, or a Nervana® (e.g., “Lake Crest”) processor from Intel Corp. In at least one embodiment, inference and/or training logic 415 illustrated in FIG. 4A may be used in conjunction with central processing unit (“CPU”) hardware, graphics processing unit (“GPU”) hardware or other hardware, such as field programmable gate arrays (“FPGAs”).

FIG. 4B illustrates inference and/or training logic 415, according to at least one embodiment. In at least one embodiment, inference and/or training logic 415 may include, without limitation, hardware logic in which computational resources are dedicated or otherwise exclusively used in conjunction with weight values or other information corresponding to one or more layers of neurons within a neural network. In at least one embodiment, inference and/or training logic 415 illustrated in FIG. 4B may be used in conjunction with an application-specific integrated circuit (ASIC), such as TensorFlow® Processing Unit from Google, an inference processing unit (IPU) from Graphcore™, or a Nervana® (e.g., “Lake Crest”) processor from Intel Corp. In at least one embodiment, inference and/or training logic 415 illustrated in FIG. 4B may be used in conjunction with central processing unit (CPU) hardware, graphics processing unit (GPU) hardware or other hardware, such as field programmable gate arrays (FPGAs). In at least one embodiment, inference and/or training logic 415 includes, without limitation, code and/or data storage 401 and code and/or data storage 405, which may be used to store code (e.g., graph code), weight values and/or other information, including bias values, gradient information, momentum values, and/or other parameter or hyperparameter information. In at least one embodiment illustrated in FIG. 4B, each of code and/or data storage 401 and code and/or data storage 405 is associated with a dedicated computational resource, such as computational hardware 402 and computational hardware 406, respectively. In at least one embodiment, each of computational hardware 402 and computational hardware 406 comprises one or more ALUs that perform mathematical functions, such as linear algebraic functions, only on information stored in code and/or data storage 401 and code and/or data storage 405, respectively, result of which is stored in activation storage 420.

In at least one embodiment, each of code and/or data storage 401 and 405 and corresponding computational hardware 402 and 406, respectively, correspond to different layers of a neural network, such that resulting activation from one storage/computational pair 401/402 of code and/or data storage 401 and computational hardware 402 is provided as an input to a next storage/computational pair 405/406 of code and/or data storage 405 and computational hardware 406, in order to mirror a conceptual organization of a neural network. In at least one embodiment, each of storage/computational pairs 401/402 and 405/406 may correspond to more than one neural network layer. In at least one embodiment, additional storage/computation pairs (not shown) subsequent to or in parallel with storage/computation pairs 401/402 and 405/406 may be included in inference and/or training logic 415.

### Neural Network Training and Deployment

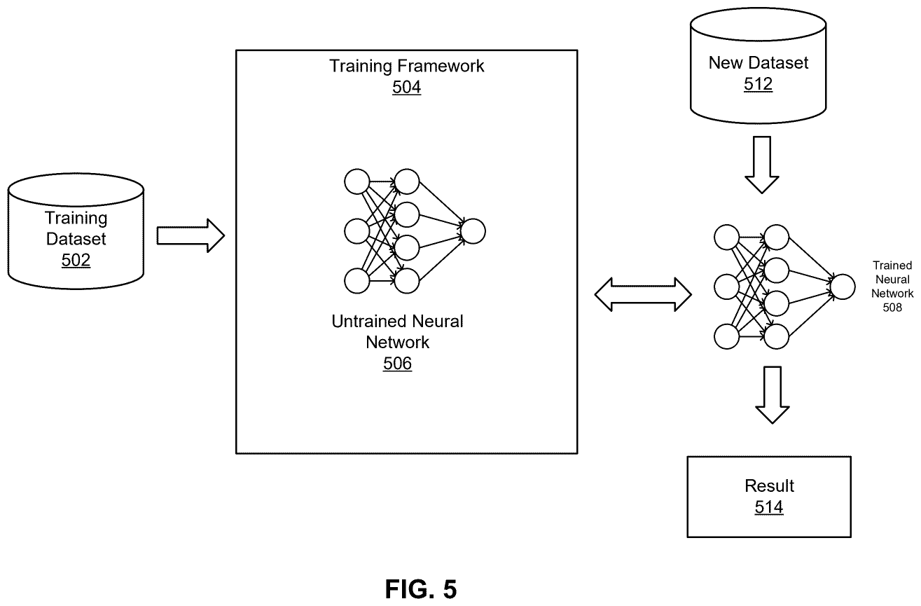

FIG. 5 illustrates training and deployment of a deep neural network, according to at least one embodiment. In at least one embodiment, untrained neural network 506 is trained using a training dataset 502. In at least one embodiment, training framework 504 is a PyTorch framework, whereas in other embodiments, training framework 504 is a TensorFlow, Boost, Caffe, Microsoft Cognitive Toolkit/CNTK, MXNet, Chainer, Keras, Deeplearning4j, or other training framework. In at least one embodiment, training framework 504 trains an untrained neural network 506 and enables it to be trained using processing resources described herein to generate a trained neural network 508. In at least one embodiment, weights may be chosen randomly or by pre-training using a deep belief network. In at least one embodiment, training may be performed in either a supervised, partially supervised, or unsupervised manner.

In at least one embodiment, untrained neural network 506 is trained using supervised learning, wherein training dataset 502 includes an input paired with a desired output for an input, or where training dataset 502 includes input having a known output and an output of neural network 506 is manually graded. In at least one embodiment, untrained neural network 506 is trained in a supervised manner and processes inputs from training dataset 502 and compares resulting outputs against a set of expected or desired outputs. In at least one embodiment, errors are then propagated back through untrained neural network 506. In at least one embodiment, training framework 504 adjusts weights that control untrained neural network 506. In at least one embodiment, training framework 504 includes tools to monitor how well untrained neural network 506 is converging towards a model, such as trained neural network 508, suitable to generating correct answers, such as in result 514, based on input data such as a new dataset 512. In at least one embodiment, training framework 504 trains untrained neural network 506 repeatedly while adjust weights to refine an output of untrained neural network 506 using a loss function and adjustment algorithm, such as stochastic gradient descent. In at least one embodiment, training framework 504 trains untrained neural network 506 until untrained neural network 506 achieves a desired accuracy. In at least one embodiment, trained neural network 508 can then be deployed to implement any number of machine learning operations.

In at least one embodiment, untrained neural network 506 is trained using unsupervised learning, wherein untrained neural network 506 attempts to train itself using unlabeled data. In at least one embodiment, unsupervised learning training dataset 502 will include input data without any associated output data or “ground truth” data. In at least one embodiment, untrained neural network 506 can learn groupings within training dataset 502 and can determine how individual inputs are related to untrained dataset 502. In at least one embodiment, unsupervised training can be used to generate a self-organizing map in trained neural network 508 capable of performing operations useful in reducing dimensionality of new dataset 512. In at least one embodiment, unsupervised training can also be used to perform anomaly detection, which allows identification of data points in new dataset 512 that deviate from normal patterns of new dataset 512.

In at least one embodiment, semi-supervised learning may be used, which is a technique in which in training dataset 502 includes a mix of labeled and unlabeled data. In at least one embodiment, training framework 504 may be used to perform incremental learning, such as through transferred learning techniques. In at least one embodiment, incremental learning enables trained neural network 508 to adapt to new dataset 512 without forgetting knowledge instilled within trained neural network 508 during initial training.

In at least one embodiment, training framework 504 is a framework processed in connection with a software development toolkit such as an OpenVINO (Open Visual Inference and Neural network Optimization) toolkit. In at least one embodiment, an OpenVINO toolkit is a toolkit such as those developed by Intel Corporation of Santa Clara, CA.

In at least one embodiment, OpenVINO is a toolkit for facilitating development of applications, specifically neural network applications, for various tasks and operations, such as human vision emulation, speech recognition, natural language processing, recommendation systems, and/or variations thereof. In at least one embodiment, OpenVINO supports neural networks such as convolutional neural networks (CNNs), recurrent and/or attention-based neural networks, and/or various other neural network models. In at least one embodiment, OpenVINO supports various software libraries such as OpenCV, OpenCL, and/or variations thereof.

In at least one embodiment, OpenVINO supports neural network models for various tasks and operations, such as classification, segmentation, object detection, face recognition, speech recognition, pose estimation (e.g., humans and/or objects), monocular depth estimation, image inpainting, style transfer, action recognition, colorization, and/or variations thereof.

In at least one embodiment, OpenVINO comprises one or more software tools and/or modules for model optimization, also referred to as a model optimizer. In at least one embodiment, a model optimizer is a command line tool that facilitates transitions between training and deployment of neural network models. In at least one embodiment, a model optimizer optimizes neural network models for execution on various devices and/or processing units, such as a GPU, CPU, PPU, GPGPU, and/or variations thereof. In at least one embodiment, a model optimizer generates an internal representation of a model, and optimizes said model to generate an intermediate representation. In at least one embodiment, a model optimizer reduces a number of layers of a model. In at least one embodiment, a model optimizer removes layers of a model that are utilized for training. In at least one embodiment, a model optimizer performs various neural network operations, such as modifying inputs to a model (e.g., resizing inputs to a model), modifying a size of inputs of a model (e.g., modifying a batch size of a model), modifying a model structure (e.g., modifying layers of a model), normalization, standardization, quantization (e.g., converting weights of a model from a first representation, such as floating point, to a second representation, such as integer), and/or variations thereof.

In at least one embodiment, OpenVINO comprises one or more software libraries for inferencing, also referred to as an inference engine. In at least one embodiment, an inference engine is a C++ library, or any suitable programming language library. In at least one embodiment, an inference engine is utilized to infer input data. In at least one embodiment, an inference engine implements various classes to infer input data and generate one or more results. In at least one embodiment, an inference engine implements one or more API functions to process an intermediate representation, set input and/or output formats, and/or execute a model on one or more devices.

In at least one embodiment, OpenVINO provides various abilities for heterogeneous execution of one or more neural network models. In at least one embodiment, heterogeneous execution, or heterogeneous computing, refers to one or more computing processes and/or systems that utilize one or more types of processors and/or cores. In at least one embodiment, OpenVINO provides various software functions to execute a program on one or more devices. In at least one embodiment, OpenVINO provides various software functions to execute a program and/or portions of a program on different devices. In at least one embodiment, OpenVINO provides various software functions to, for example, run a first portion of code on a CPU and a second portion of code on a GPU and/or FPGA. In at least one embodiment, OpenVINO provides various software functions to execute one or more layers of a neural network on one or more devices (e.g., a first set of layers on a first device, such as a GPU, and a second set of layers on a second device, such as a CPU).

In at least one embodiment, OpenVINO includes various functionality similar to functionalities associated with a CUDA programming model, such as various neural network model operations associated with frameworks such as TensorFlow, PyTorch, and/or variations thereof. In at least one embodiment, one or more CUDA programming model operations are performed using OpenVINO. In at least one embodiment, various systems, methods, and/or techniques described herein are implemented using OpenVINO.

Other variations are within spirit of present disclosure. Thus, while disclosed techniques are susceptible to various modifications and alternative constructions, certain illustrated embodiments thereof are shown in drawings and have been described herein in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

In at least one embodiment, an arithmetic logic unit is a set of combinational logic circuitry that takes one or more inputs to produce a result. In at least one embodiment, an arithmetic logic unit is used by a processor to implement mathematical operation such as addition, subtraction, or multiplication. In at least one embodiment, an arithmetic logic unit is used to implement logical operations such as logical AND/OR or XOR. In at least one embodiment, an arithmetic logic unit is stateless, and made from physical switching components such as semiconductor transistors arranged to form logical gates. In at least one embodiment, an arithmetic logic unit may operate internally as a stateful logic circuit with an associated clock. In at least one embodiment, an arithmetic logic unit may be constructed as an asynchronous logic circuit with an internal state not maintained in an associated register set. In at least one embodiment, an arithmetic logic unit is used by a processor to combine operands stored in one or more registers of the processor and produce an output that can be stored by the processor in another register or a memory location.

In at least one embodiment, as a result of processing an instruction retrieved by the processor, the processor presents one or more inputs or operands to an arithmetic logic unit, causing the arithmetic logic unit to produce a result based at least in part on an instruction code provided to inputs of the arithmetic logic unit. In at least one embodiment, the instruction codes provided by the processor to the ALU are based at least in part on the instruction executed by the processor. In at least one embodiment combinational logic in the ALU processes the inputs and produces an output which is placed on a bus within the processor. In at least one embodiment, the processor selects a destination register, memory location, output device, or output storage location on the output bus so that clocking the processor causes the results produced by the ALU to be sent to the desired location.

In the scope of this application, the term arithmetic logic unit, or ALU, is used to refer to any computational logic circuit that processes operands to produce a result. For example, in the present document, the term ALU can refer to a floating point unit, a DSP, a tensor core, a shader core, a coprocessor, or a CPU.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.

### Example Clauses

Implementation details of various embodiments of the present disclosure are described in the following numbered clauses
1. In some embodiments, a method comprises receiving a request to generate a molecular structure, the request including an initial structure, atom type information, and bond type information for the molecular structure; generating, using a transformer layer of a generative artificial intelligence model, a fused input feature based on embedding representations of the initial structure, the atom type information, and the bond type information; generating, using the transformer layer of the generative artificial intelligence model, an intermediate predicted molecular structure for the fused input feature; refining, using a graph neural network-based layer in the generative artificial intelligence model, the intermediate predicted molecular structure into the molecular structure; and outputting the molecular structure for synthesis and experimental evaluation.
2. The method of clause 1, further comprising generating the fused input feature comprises: generating an embedding representation of the initial structure, an embedding representation of the atom type information, and an embedding representation of the bond type information; wherein generating the fused input feature comprises aggregating the embedding representation of the initial structure, the embedding representation of the atom type information, and the embedding representation of the bond type information into a plurality of input tokens.
3. The method of clause 2, wherein the fused input feature is further based on a time variable associated with a number of inferencing rounds previously performed with respect to the request.
4. The method of any of clauses 2 or 3, wherein the embedding representation of the initial structure, the embedding representation of the atom type information, and the embedding representation of the bond type information is generated based on a feedforward neural network.
5. The method of any of clauses 1 through 4, wherein the atom type information comprises elemental and charge information for each atom in the molecular structure, and wherein the bond type information comprises a bond adjacency matrix.
6. The method of any of clauses 1 through 5, further comprising: receiving feedback data based on results of the experimental evaluation of the molecular structure; and updating one or more parameters of the generative artificial intelligence model based on the feedback data to improve accuracy of subsequent molecular structure generation.
7. The method of any of clauses 1 through 6, wherein the transformer layer of the generative artificial intelligence model comprises a diffusion layer configured to generate the intermediate predicted molecular structure based on denoising a noisy representation of the molecular structure.
8. The method of any of clauses 1 through 7, wherein the transformer layer of the generative artificial intelligence model comprises a flow-matching layer.
9. The method of any of clauses 1 through 8, wherein the outputted molecular structure comprises a three-dimensional conformer and a two-dimensional graph representation of the three-dimensional structure.
10. The method of any of clauses 1 through 9, wherein the graph neural network-based layer in the generative artificial intelligence model is configured to refine the intermediate predicted molecular structure based on positional update and a cross-product update.
11. A processing system, comprising: at least one memory having executable instructions stored thereon; and one or more processors configured to execute the operations of any of clauses 1 through 10.
12. A processing system, comprising means for performing the operations of any of clauses 1 through 10.
13. A non-transitory computer readable medium having executable instructions stored thereon which, when executed by one or more processors, performs the operations of any of clauses 1 through 10.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

What is claimed is:

1. A processor-implemented method, comprising:
receiving a request to generate a molecular structure, the request including an initial structure, atom type information, and bond type information for the molecular structure;
generating, using a transformer layer of a generative artificial intelligence model, a fused input feature based on embedding representations of the initial structure, the atom type information, and the bond type information;
generating, using the transformer layer of the generative artificial intelligence model, an intermediate predicted molecular structure for the fused input feature;
refining, using a graph neural network-based layer in the generative artificial intelligence model, the intermediate predicted molecular structure into the molecular structure; and
outputting the molecular structure for synthesis and experimental evaluation.

2. The method of claim 1, further comprising:
generating an embedding representation of the initial structure, an embedding representation of the atom type information, and an embedding representation of the bond type information;
wherein generating the fused input feature comprises aggregating the embedding representation of the initial structure, the embedding representation of the atom type information, and the embedding representation of the bond type information into a plurality of input tokens.

3. The method of claim 2, wherein the fused input feature is further based on a time variable associated with a number of inferencing rounds previously performed with respect to the request.

4. The method of claim 2, wherein the embedding representation of the initial structure, the embedding representation of the atom type information, and the embedding representation of the bond type information is generated based on a feedforward neural network.

5. The method of claim 1, wherein the atom type information comprises elemental and charge information for each atom in the molecular structure, and wherein the bond type information comprises a bond adjacency matrix.

6. The method of claim 1, further comprising:
receiving feedback data based on results of the experimental evaluation of the molecular structure; and
updating one or more parameters of the generative artificial intelligence model based on the feedback data to improve accuracy of subsequent molecular structure generation.

7. The method of claim 1, wherein the transformer layer of the generative artificial intelligence model comprises a diffusion layer configured to generate the intermediate predicted molecular structure based on denoising a noisy representation of the molecular structure.

8. The method of claim 1, wherein the transformer layer of the generative artificial intelligence model comprises a flow-matching layer.

9. The method of claim 1, wherein the outputted molecular structure comprises a three-dimensional conformer and a two-dimensional graph representation of the three-dimensional structure.

10. The method of claim 1, wherein the graph neural network-based layer in the generative artificial intelligence model refines the intermediate predicted molecular structure based on positional update and a cross-product update.

11. A processing system, comprising:
at least one memory having executable instructions stored thereon; and
one or more processors configured to execute the executable instructions to cause the processing system to:
receive a request to generate a molecular structure, the request including an initial structure, atom type information, and bond type information for the molecular structure;
generate, using a transformer layer of a generative artificial intelligence model, a fused input feature based on embedding representations of the initial structure, the atom type information, and the bond type information;
generate, using the transformer layer of the generative artificial intelligence model, an intermediate predicted molecular structure for the fused input feature;
refine, using a graph neural network-based layer in the generative artificial intelligence model, the intermediate predicted molecular structure into the molecular structure; and
output the molecular structure for use in a drug discovery pipeline.

12. The processing system of claim 11, wherein the one or more processors are configured to cause the processing system to:
generate an embedding representation of the initial structure, an embedding representation of the atom type information, and an embedding representation of the bond type information;
wherein to generate the fused input feature, the one or more processors are configured to cause the processing system to aggregate the embedding representation of the initial structure, the embedding representation of the atom type information, and the embedding representation of the bond type information into a plurality of input tokens.

13. The processing system of claim 12, wherein the fused input feature is further based on a time variable associated with a number of inferencing rounds previously performed with respect to the request.

14. The processing system of claim 12, wherein the embedding representation of the initial structure, the embedding representation of the atom type information, and the embedding representation of the bond type information is generated based on a feedforward neural network.

15. The processing system of claim 11, wherein the atom type information comprises elemental and charge information for each atom in the molecular structure, and wherein the bond type information comprises a bond adjacency matrix.

16. The processing system of claim 11, wherein the one or more processors are further configured to cause the processing system to:
receive feedback data based on results of the experimental evaluation of the molecular structure; and
update one or more parameters of the generative artificial intelligence model based on the feedback data to improve accuracy of subsequent molecular structure generation.

17. The processing system of claim 11, wherein the transformer layer of the generative artificial intelligence model comprises a diffusion layer configured to generate the intermediate predicted molecular structure based on denoising a noisy representation of the molecular structure.

18. The processing system of claim 11, wherein the transformer layer of the generative artificial intelligence model comprises a flow-matching layer.

19. The processing system of claim 11, wherein the outputted molecular structure comprises a three-dimensional conformer and a two-dimensional graph representation of the three-dimensional structure.

20. The processing system of claim 11, wherein the graph neural network-based layer in the generative artificial intelligence model refines the intermediate predicted molecular structure based on positional update and a cross-product update.
